(19) United States
(12) Patent Application Publication
(10) Pub. No.: US 2024/0008724 A1
(43) Pub. Date: Jan. 11, 2024

(54) HYBRID WATER RESERVOIR FOR AN ENDOSCOPE

(71) Applicant: Boston Scientific Scimed, Inc., Maple Grove, MN (US)

(72) Inventors: Ryan V. Wales, Northborough, MA (US); Colby Harris, Norfolk, MA (US); Ryan Vincent William Pollock, Leominster, MA (US); Scott E. Brechbiel, Acton, MA (US); Kurt Nicholas Robakiewicz, Upton, MA (US); Laura Elizabeth Christakis, Framingham, MA (US); Paul Smith, Smithfield, RI (US); Jeff Gray, Sudbury, MA (US); John B. Golden, Norton, MA (US)

(73) Assignee: Boston Scientific Scimed, Inc., Maple Grove, MN (US)

(21) Appl. No.: 18/348,992

(22) Filed: Jul. 7, 2023

Related U.S. Application Data
(60) Provisional application No. 63/359,752, filed on Jul. 8, 2022.

(51) Int. Cl. A61B 1/015

(52) U.S. Cl. CPC A61B 1/015

(57) ABSTRACT

Methods and systems for refilling a container during an endoscopic procedure. An illustrative container may comprise a container configured to contain a fluid, a water supply tube including a first end, a second end, and a first lumen extending therethrough and in fluid communication with the bottom portion of the container, a gas supply tube including a first end, a second end, and a second lumen extending therethrough and in operative fluid communication with container, a manifold configured to couple the water supply tube and the gas supply tube to the container and including a first end positioned interior to the container and a second end positioned exterior to the container, and a port positioned adjacent to the top portion of the container. The port may be configured to be in selective fluid communication with an interior of the container.

### CROSS-REFERENCE TO RELATED APPLICATIONS

This application claims the benefit of U.S. Prov. Pat. App. No. 63/359,752, filed Jul. 8, 2022, titled HYBRID WATER RESERVOIR FOR AN ENDOSCOPE, which is incorporated herein by reference.

### FIELD

This disclosure relates generally to medical fluid containers and tubing assemblies and methods, and particularly to a container and tube assembly to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or alternative source are used to increase the pressure within a fluid bottle which either insufflates the working lumen or wash the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container arranged and configured to couple to an endoscope for use in an endoscopic procedure may comprise a container configured to contain a fluid, the container having a bottom portion and a top portion; a manifold including a first end positioned interior to the container and a second end positioned exterior to the container, the manifold configured to couple a water supply tube and a gas supply tube to the container, and a port positioned adjacent to the top portion of the container. The port may be configured to be in selective fluid communication with an interior of the container.

Alternatively or additionally to any of the examples above, in another example, the container may further comprise the water supply tube and the gas supply tube. The water supply tube may include a first end, a second end, and a first lumen extending therethrough. The first lumen may be in fluid communication with the bottom of the container and the second end of the water supply tube may be positioned external to the container. The gas supply tube may include a first end, a second end, and a second lumen extending therethrough. The second lumen may be in operative fluid communication with the interior the container and the second end of the gas supply tube may be positioned external to the container.

Alternatively or additionally to any of the examples above, in another example, the first end of the manifold may comprise an air outlet and a fluid inlet.

Alternatively or additionally to any of the examples above, in another example, a first lumen of the water supply tube may be fluidly coupled to the fluid inlet.

Alternatively or additionally to any of the examples above, in another example, the air outlet may comprise a one-way valve.

Alternatively or additionally to any of the examples above, in another example, the second end of the manifold may comprise an air inlet.

Alternatively or additionally to any of the examples above, in another example, a first end of the gas supply tube may be configured to be coupled to the air inlet of the manifold.

Alternatively or additionally to any of the examples above, in another example, the water supply tube may be configured to coaxially extend through the air inlet of the manifold to the fluid inlet.

Alternatively or additionally to any of the examples above, in another example, the manifold may further comprise at least one laterally extending protrusion.

Alternatively or additionally to any of the examples above, in another example, the at least one laterally extending protrusion may be positioned interior to the container.

Alternatively or additionally to any of the examples above, in another example, the at least one laterally extending protrusion may be adhered to the interior of the container.

Alternatively or additionally to any of the examples above, in another example, the container may further comprise a cap removably coupled to the port.

Alternatively or additionally to any of the examples above, in another example, the cap may comprise a tapered plug.

Alternatively or additionally to any of the examples above, in another example, the cap may comprise a self-sealing cap.

Alternatively or additionally to any of the examples above, in another example, the cap may comprise a self-sealing valve.

Alternatively or additionally to any of the examples above, in another example, the container may further comprise a hanging hook.

In another example, a container and tube set arranged and configured to couple to an endoscope for use in an endoscopic procedure may comprise a vessel, a first chamber disposed within the vessel and configured to contain a fluid, an inflatable bladder having an inlet and disposed within the vessel, a water outlet in fluid communication with the first chamber, a gas inlet in fluid communication with the inflatable bladder, and a port positioned adjacent to the top portion of the first chamber. The port may be configured to be in selective fluid communication with an interior of the first chamber.

Alternatively or additionally to any of the examples above, in another example, the water outlet may comprise a water supply tube including a first end, a second end, and a first lumen extending therethrough. The first lumen may be in fluid communication with the first chamber and the second end of the water supply tube may be positioned external to the vessel. The gas inlet may comprise a gas supply tube including a first end, a second end, and a second lumen extending therethrough. The second lumen may be in operative communication with the inlet of the inflatable bladder and the second end of the gas supply tube may be positioned external to the container.

Alternatively or additionally to any of the examples above, in another example, the container and tube set may further comprise a one-way valve disposed in the inlet of the inflatable bladder.

Alternatively or additionally to any of the examples above, in another example, the one-way valve may be removable.

Alternatively or additionally to any of the examples above, in another example, the inflatable bladder may be configured to expand to expel fluid from the first chamber.

Alternatively or additionally to any of the examples above, in another example, the container and tube set may further comprise a cap removably coupled to the port.

Alternatively or additionally to any of the examples above, in another example, the cap may comprise a tapered plug.

Alternatively or additionally to any of the examples above, in another example, the cap may comprise a self-sealing cap.

Alternatively or additionally to any of the examples above, in another example, the cap may comprise a self-sealing valve.

In another example, a container arranged and configured to couple to an endoscope for use in an endoscopic procedure may comprise a container configured to contain a fluid, the container having a bottom portion and a top portion, a water supply tube including a first end, a second end, and a first lumen extending therethrough, wherein the first lumen is in fluid communication with the bottom portion of the container and the second end of the water supply tube is positioned external to the container, a gas supply tube including a first end, a second end, and a second lumen extending therethrough, wherein the second lumen is in operative fluid communication with the container and the second end of the gas supply tube is positioned external to the container, and a port positioned adjacent to the top portion of the container, wherein the port is configured to be in selective fluid communication with the container and wherein the port is configured to couple directly to a water bottle.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3A depicts an endoscope system with endoscope, light source, water reservoir, and tubing assembly for hybrid air, lens wash and irrigation fluid delivery, wherein the system is activated to deliver air to atmosphere;

FIG. 3B depicts the endoscope system of FIG. 3A, wherein the system is activated to deliver air to a patient through the patient end of the endoscope;

FIG. 3C depicts the endoscope system of FIG. 3A, wherein the system is activated to deliver lens wash fluid through the patient end of the endoscope;

FIG. 3D depicts the endoscope system of FIG. 3A, wherein the system is activated to deliver irrigation fluid through the patient end of the endoscope;

FIG. 4 depicts a hybrid endoscope system including a video processing unit, connector portion, peristaltic irrigation pump, water reservoir and top, coaxial gas and lens wash supply tubing, upstream and downstream irrigation supply tubing, and alternative gas supply tubing;

FIG. 5 depicts a perspective view of an illustrative refillable fluid reservoir;

FIG. 6 depicts a side view of the reservoir of FIG. 5 with an alternative port;

FIG. 7 depicts a perspective view of another illustrative refillable fluid reservoir;

FIG. 8A depicts a schematic side view of another illustrative refillable fluid reservoir in a first configuration;

FIG. 8B depicts a schematic side view of the illustrative reservoir of FIG. 8A in a second configuration;

FIG. 9A depicts a perspective view of an illustrative manifold configured to couple a lens wash or shared water supply tube and a gas or shared gas supply tube to a water reservoir;

FIG. 9B depicts an alternative perspective view of the illustrative manifold of FIG. 9A;

FIG. 9C depicts a cross-sectional view of the illustrative manifold of FIG. 9A, taken at line 9C-9C, and including a gas supply tube and a water supply tube;

FIG. 10A depicts illustrative coupling mechanisms for securing a reservoir relative to an endoscope tower;

FIG. 10B depicts another illustrative coupling mechanism for securing a reservoir relative to an endoscope tower; and

FIG. 10C depicts additional illustrative coupling mechanisms for securing a reservoir relative to an endoscope tower.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or alternative source are used to increase the pressure within a fluid bottle which either insufflates the working lumen or wash the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day which may introduce multiple opportunities for contamination to the tube set by either contacting non-sterile surfaces or dropping the tubing on the floor. Disclosed herein are methods and systems to reduce or eliminate the need to disconnect the tube set and use a second bottle.

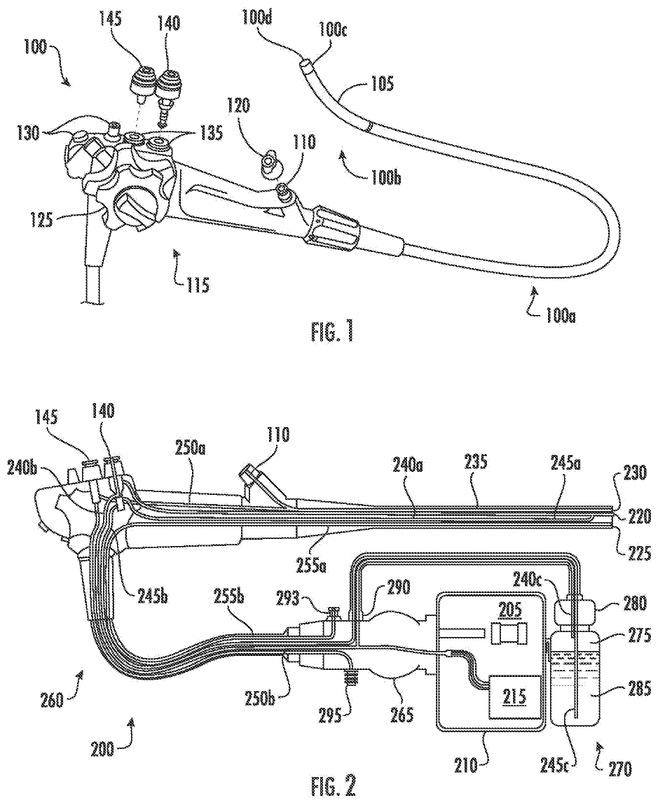

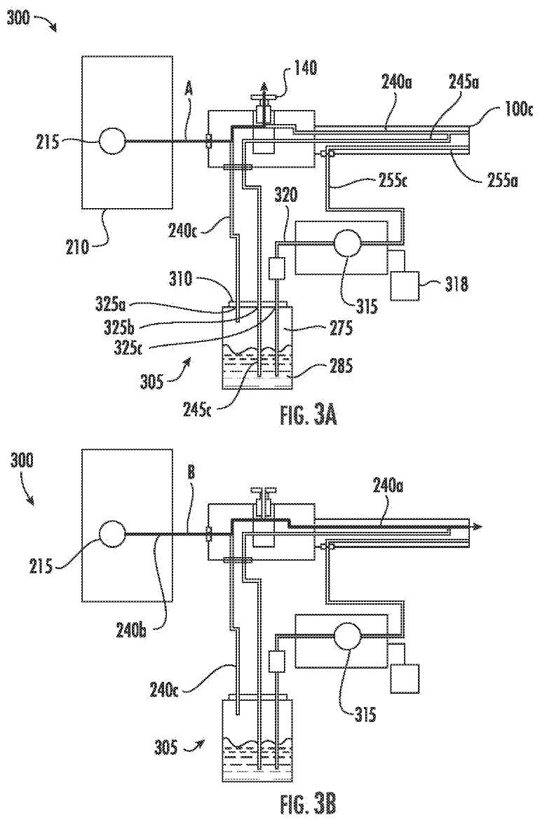

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connection portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

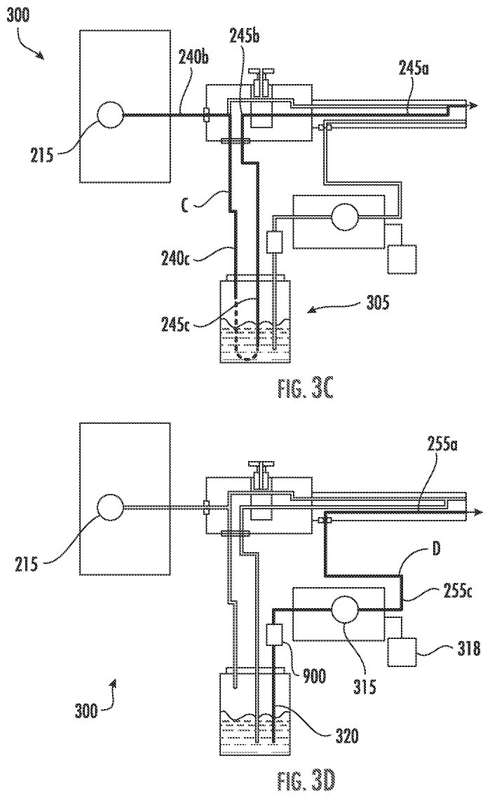

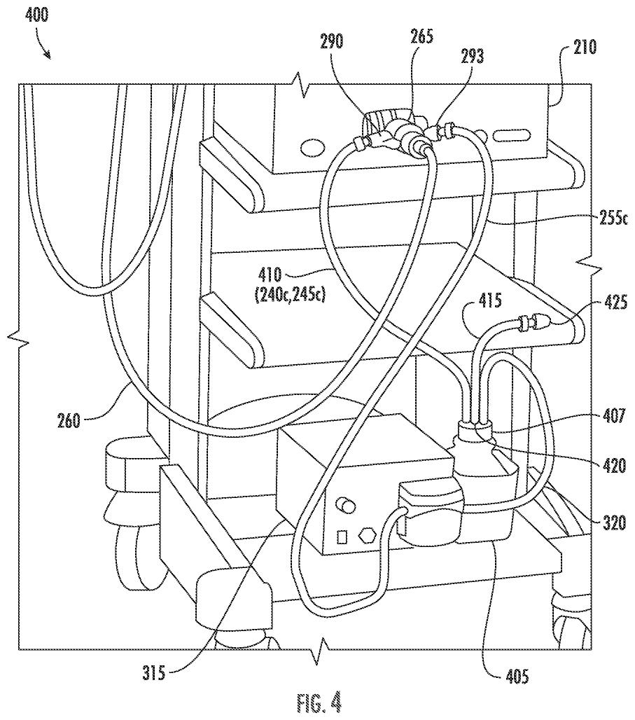

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream irrigation tubing 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 240c, upstream irrigation supply tubing 320, and/or downstream irrigation supply tubing 255c to help prevent backflow of water from either or both of the lens wash and irrigation tubing in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 240c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash 240c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 240c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 240c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to delivery water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, and alternative gas supply tubing 415 (e.g., CO₂). A length of the alternative gas supply tubing 415 passes from one end positioned in the gas gap 275 between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivery gas through alternative gas (e.g., CO₂) supply tubing 415. The irrigation function may be accomplished in a similar manner as the operation described above with respect to FIG. 3D.

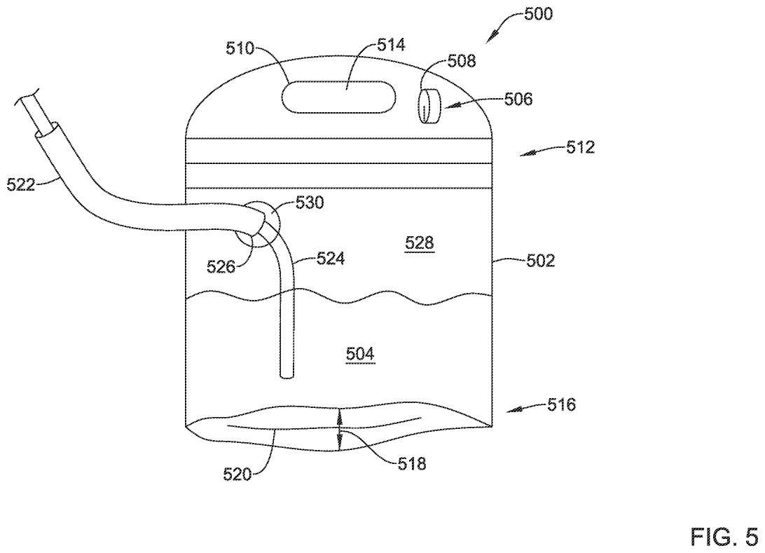

As described above, it may be desirable to reduce opportunities for contamination to the tube set 240c, 245c, 320, 410, 415 during replacement of the water reservoir by providing a refillable and/or larger volume water reservoir 270, 305, 405. FIG. 5 depicts a perspective view of an illustrative refillable fluid reservoir 500. The reservoir 500 may be configured to be used in an endoscopic system and includes components similar to the endoscope and endoscope systems described with regard to FIGS. 1-4; however, not all features may be described or shown here if not pertinent to the fluid circuit of the system. The reservoir 500 includes a container 502 configured to hold a fluid 504. The container 502 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In other embodiments, the reservoir 500 may be formed from a rigid or semi-rigid material. In some embodiments, the container 502 may be entirely translucent, entirely opaque, or combinations thereof. The reservoir 500 may further include a port 506 having a removable cap 508. The cap 508 may be formed from a rigid material and may be configured to form a fluid tight seal with the port 506. The cap 508 may be configured to threadably engage the port 506, form a friction fit with the port 506, form a snap fit with the port 506, or otherwise releasably engage the port 506. In some embodiments, the cap 508 may be a self-sealing one-way valve. In other embodiments, the cap 508 may be formed from a self-healing material. For example, a needle may be used to puncture a self-healing material, providing access to an interior of the container 502, and once the needle is removed, the hole formed by the needle is sealed without user intervention. In some examples, the port 506 and/or cap 508 may be formed from polyethylene terephthalate (PET), polypropylene (PP), etc. Portions of the port 506 may extend into the container 502. The removable cap 508 may be removed to place a fluid source in selective fluid communication with the container 502 and allow fluid to be poured through a lumen of the port 506 and into the container 502. In the case of a cap 508 formed from a self-healing material, a needle may be used to puncture the cap 508 and place a fluid source in selective fluid communication with the container 502 and allow fluid to be poured into the container 502.

The reservoir 500 may include a carrying handle or hanging hook 510 positioned adjacent to a top portion 512 thereof. The handle 510 may define an opening or through hole 514 for receiving a hand or hook therethrough to carry or otherwise support the reservoir 500. In some embodiments, the handle 510 may be configured to couple to a hook or other mechanism on the endoscope tower. In some cases, the reservoir 500 may be coupled to the tower via hanging loop or hook and loop closures. This may elevate the reservoir to reduce footprint on floor of the procedure room and improve ergonomics since the user may no longer need to bend down to the floor to interface with the reservoir. In some cases, the handle 510 may be configured to provide a more ergonomic grip for the user. It is contemplated that the handle 510 may be formed from a similar material as the cap 508 or the container 502, as desired. In some examples, the handle 510 may be formed from polyethylene terephthalate (PET), polypropylene (PP), etc.

The reservoir 500 may be movable between a collapsed storage configuration (not explicitly shown) and an expanded use configuration (FIG. 5). In the expanded use configuration, the reservoir 500 may increase in width 518 (e.g., into/out of the drawing page) from the top portion 512 towards the bottom portion 516. In the use configuration, the bottom portion 516 may have a width that allows the reservoir 500 to remain upright without user intervention. The bottom portion 516 may include folds or pleats 520 that allow the bottom portion 516 to fold or collapse. In the collapsed storage configuration, the top portion 512 and the bottom portion 516 may have a similar width 518 which allows the reservoir 500 to lay substantially flat such that the reservoir 500 may be stacked with other fluid reservoirs 500. In other examples, the reservoir 500 may be rolled or folded to reduce the amount of storage space it occupies. In some cases, since the reservoir 500 is sealed or capable of being sealed, a vacuum may be pulled during packaging of the reservoir 500 to further reduce the storage space required to store the reservoir 500. In some embodiments, the reservoir 500 may be stored inside out such that the tubing, manifold, fittings and/or other components are within the container 502. When the reservoir 500 is taken from storage for use, the entire inside of the reservoir 500 may be pulled through the port 506 and the tubes connected to the connector portion 265 of the umbilical 260. The reservoir 500 may be stored in sterile packaging to maintain sterility of the tubing 536, 538 and interior surface of the container 502.

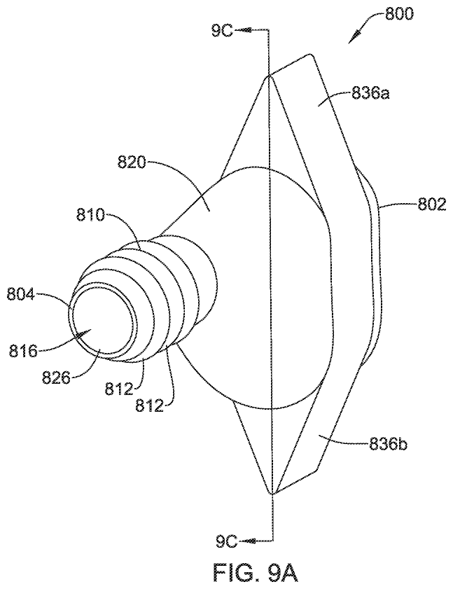

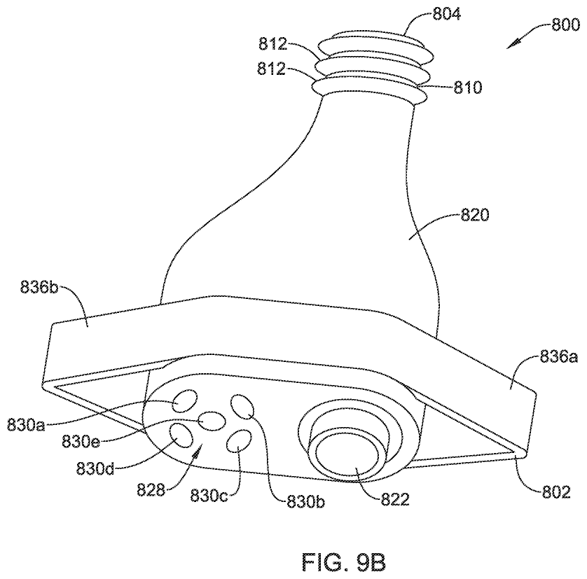

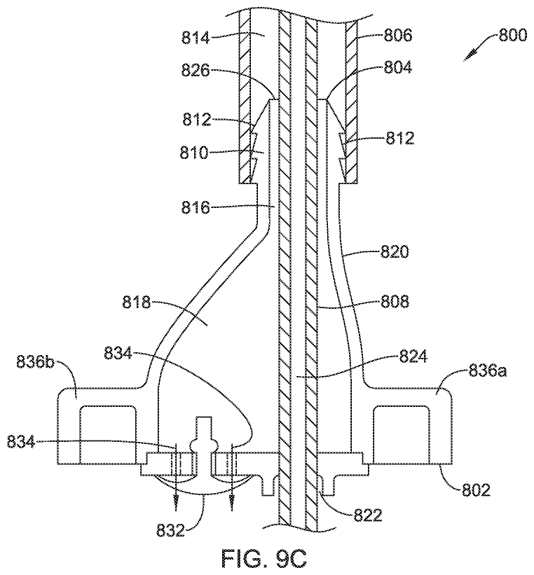

The reservoir 500 may be connected in fluid communication with a tubing manifold (not explicitly shown) via a shared length of gas supply/alternate gas supply tubing (or gas supply tubing) 522 and a lens wash supply/irrigation supply tubing (or water supply tubing) 524. The shared gas supply tubing 522 extends from a second end external to the reservoir 500 through a reservoir opening 526 in the top portion 512 of the container 502. The shared gas supply tubing 522 may terminate within a reservoir gap 528, at or below the opening 526, but not extending into the remaining fluid 504 in the container 502. However, in some cases, the gas supply tubing 522 may extend into the fluid 504. For example, the opening 526 may be at a bottom or side of the container 502 such that the shared gas supply tubing 522 terminates within the fluid 504 with gas bubbling up through the fluid 504 to pressurize the container 502. A lumen extends through the gas supply tubing 522 for receiving a flow of air and/or gas therethrough. The lumen of the gas supply tubing 522 is in operative fluid communication with the top portion 512 of the reservoir 500. The water supply tubing 524 extends from a second end external to the reservoir 500 through the reservoir opening 526, terminating in a first end within the remaining fluid 504 at or substantially at the bottom of the container 502. In some embodiments, the water supply tubing 524 may terminate at the opening 526. For example, when the opening 526 is at or adjacent to the bottom portion 526 of the container 502 a dip tube may not be required. A lumen extends through the water supply tubing 524 for receiving a flow of fluid therethrough. The lumen of the lens wash supply/irrigation supply tubing 524 is in selective operative fluid communication with the bottom portion of the container 502. In the illustrated embodiment, the gas supply tubing 522 and the water supply tubing 524 may enter the container 502 through a single or common opening 526. For example, the gas supply tubing 522 and the water supply tubing 524 may be coaxially arranged. However, this is not required. In some cases, the gas supply tubing 522 and the water supply tubing 524 may extend in a side by side arrangement or may be separately connected to the container 502 in different locations. The opening 526 may include a grommet or heat seal 530 configured to seal the container 502 about the tubing 522, 524 in a fluid and pressure tight manner. In other embodiments, a manifold, such as the manifold described with respect to FIGS. 9A-9C may be used to couple the tubing 522, 524 to the reservoir 500 in a fluid-tight manner.

A portion of a gas supply tubing 522 and a portion of lens wash supply tubing 524 may be connected in fluid communication with the endoscope at gas/lens wash connection on the connector portion 265 of the umbilical. The gas supply tubing 522 is connected in fluid communication with a gas pump (not explicitly shown) and gas feed line (not explicitly shown), and the lens wash supply tubing 524 is connected in fluid communication with lens wash feed line (not explicitly shown), within connector portion 265. In some examples, the gas supply tubing 522 may include a manifold to fluidly couple portions of the gas supply tubing 522. Similarly, the lens wash supply tubing 524 may include a manifold to fluidly couple portions of the lens wash supply tubing with the shared lens wash/irrigation (or water) supply tubing 524. While not explicitly shown, irrigation supply tubing may be coupled to the manifold, if so provided, to supply irrigation fluid from the reservoir 500. In other cases, a separate irrigation supply tube may be provided.

It is contemplated that the reservoir 500 may be filled and refilled as needed by removing the cap 508 or puncturing the cap and introducing water into the container 502. The refilling of the reservoir 500 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 500 with sterile or non-sterile water may create more flexibility and reduce the need to have as much sterile water in storage. Further, refilling the reservoir 500 via the port 506 and removable cap 508 may also remove the need to disconnect the reservoir 500 from the tubing 522, 524 throughout the day eliminating or greatly reducing the possibility of cross contamination by removing the need to replace the water container.

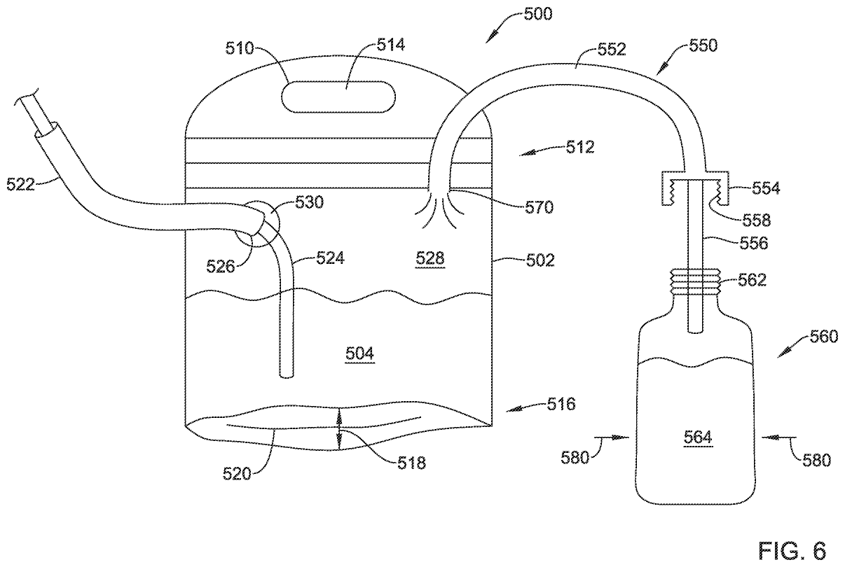

In some cases, it may be desirable to refill the reservoir 500 without having to tip or pour the fluid source. FIG. 6 depicts a side view of the reservoir 500 with an alternative port 550 that may be used with the reservoir 500 (or other reservoirs described herein) which allows both the reservoir 500 and the fluid source 560 to remain upright during filling of the reservoir 500 and/or during coupling of the fluid source 560 to the port 550. The port 550 may include an elongate tube 552 extending from an opening 570 in the container 502 to a coupling 554. In some embodiments, the elongate tube 552 may be omitted. In such an instance, the coupling 554 may be coupled directly to the reservoir 500.

In some embodiments, the elongate tube 552 may include a portion 556 that extends beyond the coupling to be disposed within the fluid source 560. It is contemplated that the extension portion 556 of the elongate tube 552 may be formed as single unitary structure with the elongate tube 552 or may be a separate component that is releasably secured to the elongate tube 552 and/or the coupling 554. In other embodiments, the port 550 may be free from the extension portion 556. The coupling 554 may include a plurality of internal threads 558 configured to engage mating threads 562 on a fluid source or bottle 560. While not explicitly shown, when the reservoir 500 is not coupled to a fluid source, a cap or plug may be engaged with the coupling 554 to provide a fluid-tight seal.

When it is desired to fill or refill the reservoir 500, the cap or plug may be removed from the coupling 554. If the extension portion 556 is provided as a separate component, the extension portion 556 may be coupled to the elongate tube 552 and/or coupling 554. The fluid source 560 may then be coupled to the coupling 554. In some cases, the user may squeeze or apply a compressive force to the fluid source 560, as shown at arrow 580. This may force fluid from the fluid source 560 and into the reservoir 500. It is contemplated that in some cases, for example, when the extension portion 556 is not provided, after coupling the fluid source 560 with the coupling 556, the fluid source 560 may be raised and inverted to pour fluid into the reservoir 500. It is contemplated that by providing a fluid-tight seal between the fluid source 560 and the port 550, unintentional spills or fluid loss may be reduced or avoided.

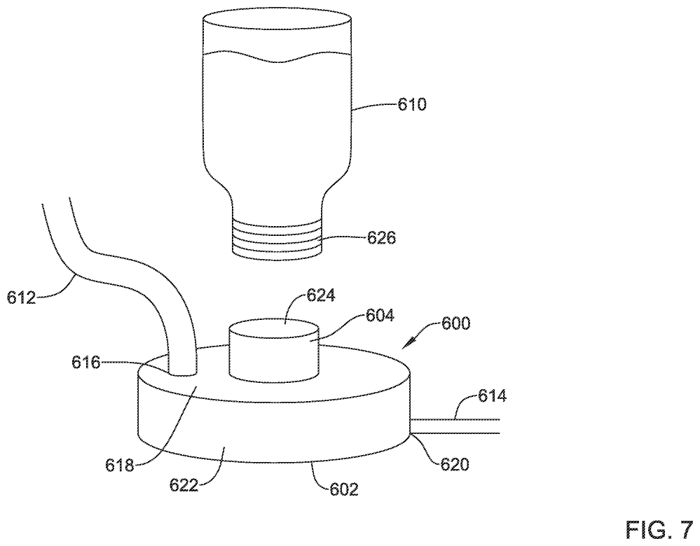

FIG. 7 depicts a perspective view of another illustrative refillable fluid reservoir 600. The reservoir 600 may be configured to be used in an endoscopic system and includes components similar to the endoscope and endoscope systems described with regard to FIGS. 1-4; however, not all features may be described or shown here if not pertinent to the fluid circuit of the system. The reservoir 600 includes a container 602 configured to hold a fluid. The container 602 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In other embodiments, the container 602 may be formed from a semi-rigid or rigid material, such as, but not limited to polyethylene terephthalate (PET), polypropylene (PP), etc. In some embodiments, the container 602 may be entirely translucent, entirely opaque, or combinations thereof.

The container 602 may be sized and shaped to hold of volume of fluid. In some cases, the volume of fluid may be approximately equal to 1 liter (e.g., the typical volume of a water bottle provided in medical procedures). In other embodiments, the container 602 may have a volume greater than 1 liter. In yet other embodiments, the container 602 may have a volume of less than one liter. It is contemplated that when the volume of the container 602 is less than 1 liter, container 602 may be coupled to a fluid source, such as, but not limited to, a water bottle 610 during a procedure. While the container 602 is illustrated as having a generally cylindrical shape, the container 602 may take other forms, as desired. It is further contemplated that the reservoir 600 may be provided as a manifold configured to interface with another reservoir to provide a means for refilling the additional reservoir.

The reservoir 600 may further include a port 604 having a removable cap or plug (not explicitly shown). The cap may be configured to form a fluid tight seal with the port 604. The cap may be configured to threadably engage the port 604, form a friction fit with the port 604, form a snap fit with the port 604, or otherwise releasably engage the port 604. In some embodiments, the cap may be a self-sealing one-way valve. In other embodiments, the cap may be formed from a self-healing material. For example, a needle may be used to puncture a self-healing material and once the needle is removed, the hole formed by the needle is sealed without user intervention. Portions of the port 604 may extend into the container 602. The removable cap may be removed to place a fluid source 610 in selective fluid communication with the container 602 and allow fluid to be poured through a lumen of the port 604 and into the container 602.

The reservoir 600 may be connected in fluid communication with a tubing manifold (not explicitly shown) via a shared gas supply/alternate gas supply tubing (or gas supply tubing) 612 and a lens wash supply/irrigation supply tubing 614. The shared gas supply tubing 612 extends from a second end external to the reservoir 600 through a reservoir opening 616 in the top portion 618 of the container 602. The shared gas supply tubing 612 may terminate within a reservoir gap, at or below the opening 616, but not extending into the remaining fluid in the container 602. However, in some cases, the gas supply tubing 612 may extend into the fluid. For example, the opening 616 may be at a bottom or side of the container 602 such that the shared gas supply tubing 612 terminates within the fluid with gas bubbling up through the fluid to pressurize the container 602. A lumen extends through the gas supply tubing 612 for receiving a flow of air and/or gas therethrough. The lumen of the gas supply tubing 612 is in operative fluid communication with the top portion 618 of the reservoir 600.

The water supply tubing 614 extends from a second end external to the reservoir 600 through a reservoir opening 620, terminating in a first end within the remaining fluid at or substantially at the bottom portion 622 of the container 602. A lumen extends through the water supply tubing 614 for receiving a flow of fluid therethrough. The lumen of the lens wash supply/irrigation supply tubing 614 is in selective operative fluid communication with the bottom portion of the container 602. In the illustrated embodiment, the gas supply tubing 612 and the water supply tubing 614 may enter/exit the container 602 through separate openings 616, 620. However, this is not required. For example, the gas supply tubing 612 and the water supply tubing 614 may be coaxially arranged and enter the container 602 through a common opening. The openings may include a grommet or heat seal configured to seal the container 602 about the tubing 612, 614 in a fluid and pressure tight manner. In other embodiments, a manifold, such as the manifold described with respect to FIGS. 9A-9C may be used to couple the tubing 612, 614 to the reservoir 600 in a fluid-tight manner.

A portion of a gas supply tubing 612 and a portion of lens wash supply tubing 614 may be connected in fluid communication with the endoscope at gas/lens wash connection on the connector portion 265 of the umbilical. The gas supply tubing 612 is connected in fluid communication with a gas pump (not explicitly shown) and gas feed line (not explicitly shown), and the lens wash supply tubing 614 is connected in fluid communication with lens wash feed line (not explicitly shown), within connector portion 265. In some examples, the gas supply tubing 612 may include a manifold to fluidly couple portions of the gas supply tubing 612. Similarly, the lens wash supply tubing 614 may include a manifold to fluidly couple portions of the lens wash supply tubing with the shared lens wash/irrigation (or water) supply tubing 614. While not explicitly shown, irrigation supply tubing may be coupled to the manifold, if so provided, to supply irrigation fluid from the reservoir 600. In other cases, a separate irrigation supply tube may be provided.

It is contemplated that the reservoir 600 may be filled and refilled as needed by removing the cap and coupling the water source 610 to the port 604. In some embodiments, the port 604 may include internal threads 624, or other coupling feature, configured to engage mating external threads 626, or other coupling feature on the water source 610. Water may then be transferred from the water source 610 to the reservoir 600. The refilling of the reservoir 600 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 600 with sterile or non-sterile water may create more flexibility and reduce the need to have as much sterile water in storage. Further, refilling the reservoir 600 via the port 604 and removable cap may also remove the need to disconnect the reservoir 600 from the tubing 612, 614 throughout the day eliminating or greatly reducing the possibility of cross contamination by removing the need to replace the water container.

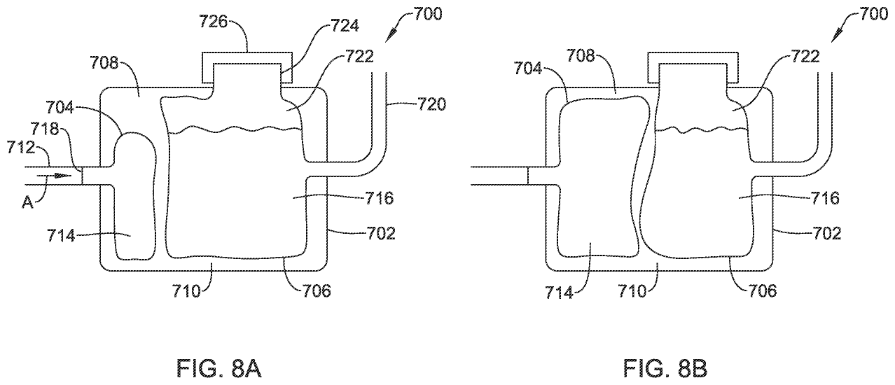

FIG. 8A depicts a schematic side view of an illustrative refillable fluid reservoir 700 in a first configuration and FIG. 8B depicts a schematic side view of the illustrative reservoir 700 of FIG. 8A in a second configuration. The reservoir 700 may be configured to be used in an endoscopic system and includes components similar to the endoscope and endoscope systems described with regard to FIGS. 1-4; however, not all features may be described or shown here if not pertinent to the fluid circuit of the system. In the illustrated embodiment, a different means for performing insufflation may be required.

The reservoir 700 may include a vessel 702 configured to hold an inflatable bladder 704 and a first fluid chamber 706. While the inflatable bladder 704 is illustrated as being located at a lateral side of the vessel 702, the inflatable bladder 704 may be positioned at other locations in the vessel 702 as desired, such as, but not limited to, along a top portion 708 of the vessel, a bottom portion 710 of the vessel 702, etc. A gas supply tubing 712 extends from a second end external to the reservoir 700 to a first end adjacent an opening in the inflatable bladder 704 such that the gas supply tubing 712 is in fluid communication with an interior or a cavity 714 of the inflatable bladder 750. A lumen extends through the gas supply tubing 712 for receiving a flow of air and/or gas therethrough. The inflatable bladder 704 fluidly isolates the air/gas received from the gas supply tubing 712 from the water 716 in the first chamber 706. The inflatable bladder 704 is configured to expand as air/gas flows into the cavity 714, along flow path A. In the absence of a positive air flow, the inflatable bladder 704 may deflate or contract. It is contemplated that the inflatable bladder 704 may include a one-way valve 718 disposed at or adjacent an inlet of the cavity 714. Examples of one-way valves include various check valves described above. The one-way valve 718 may prevent air from exiting the inflatable bladder 704 even in the absence of a positive air flow. Air flow to the interior 714 of the bladder 704 may be controlled to inflate (expand) or deflate (contract) the bladder 704, as desired. In some embodiments, the bladder 704 may be deflated by temporarily removing the one-way valve 718 or by actuating a separate vent (not explicitly shown).

A lens wash supply tube or shared water supply tube (e.g. supplies water for both lens wash and irrigation) 720 extends from a second end external to the reservoir 700 to a first end adjacent an opening in the first chamber 706 such that the water supply tubing 720 is in operative fluid communication with an interior or a cavity 722 of the first chamber 706. A lumen extends through the water supply tubing 720 for receiving a flow of fluid therethrough. As air enters the inflatable bladder 704, the bladder 704 increases in volume, as shown in FIG. 8B. It is contemplated that the vessel 702 may be formed from a material which does not expand or stretch as the inflatable bladder 704 is expanded. Thus, as the volume of the inflatable bladder 704 increases, the first chamber 706 may be compressed causing water 716 within the first chamber 706 to be expelled up the water supply tube 720 and to the endoscope for lens wash and/or irrigation.

The inflatable bladder 704 may be formed from a material which stretches, such as, but not limited to, an elastomer, to expand a volume of the inflatable bladder 704 as air is supplied thereto. The first chamber 706 may be formed from a lightweight, flexible material that does not necessarily stretch, such as, but not limited to low density polyethylene (LDPE), thermoplastic polyurethane (TPU), silicone, polyethylene terephthalate (PET), aluminum, nylon, polyethylene (PE), or combinations thereof, etc.

The first chamber 706 may further include a port 724 having a removable cap 726. The port 724 and/or cap 726 may be formed from more rigid material (relative to the first chamber 706) and may be configured to form a fluid tight seal with the port 724. The cap 726 may be configured to threadably engage the port 724, form a friction fit with the port 724, form a snap fit with the port 724, or otherwise releasably engage the port 724. In some embodiments, the cap 726 may be a self-sealing one-way valve. In other embodiments, the cap 726 may be formed from a self-healing material. For example, a needle may be used to puncture a self-healing material and once the needle is removed, the hole formed by the needle is sealed without user intervention. In some examples, the port 724 and/or cap 726 may be formed from polyethylene terephthalate (PET), polypropylene (PP), etc. Portions of the port 724 may extend into the container 502. The removable cap 726 may be removed to place a fluid source in selective fluid communication with the first chamber 706 and allow fluid to be poured through a lumen of the port 724 and into the first chamber 706

While not explicitly shown, the reservoir 700 may include a carrying handle or hanging hook. The handle may define an opening or through hole for receiving a hand or hook therethrough to carry or otherwise support the reservoir 700. In some embodiments, the handle may be configured to couple to a hook or other mechanism on the endoscope tower. In some cases, the reservoir may be coupled to the tower via hanging loop or hook and loop closures. This may elevate the reservoir to reduce footprint on floor of the procedure room and improve ergonomics since the user may no longer need to bend down to the floor to interface with the reservoir. In some cases, the handle may be configured to provide a more ergonomic grip for the user. It is contemplated that the handle may be formed from a similar material as the cap 726 or the vessel 702, as desired. In some examples, the handle may be formed from polyethylene terephthalate (PET), polypropylene (PP), etc.

A portion of a gas supply tubing 712 and a portion of water supply tubing 720 may be connected in fluid communication with the endoscope at gas/lens wash connection on the connector portion 265 of the umbilical. The gas supply tubing 712 is connected in fluid communication with a gas pump (not explicitly shown) and gas feed line (not explicitly shown), and the water supply tubing 720 is connected in fluid communication with lens wash feed line (not explicitly shown), within connector portion 265. In some examples, the gas supply tubing 712 may include a manifold to fluidly couple portions of the gas supply tubing 712. Similarly, the lens wash supply tubing 720 may include a manifold to fluidly couple portions of the lens wash supply tubing with the shared lens wash/irrigation (or water) supply tubing 720. While not explicitly shown, irrigation supply tubing may be coupled to the manifold, if so provided, to supply irrigation fluid from the reservoir 700. In other cases, a separate irrigation supply tube may be provided.

It is contemplated that the reservoir 700 may be filled and refilled as needed by removing the cap 726 and pouring water into the first chamber 706. In some examples, the inflatable bladder 704 may be deflated prior to pouring water into the first chamber 706. Deflation of the inflatable bladder 704 may be achieved by temporarily removing the one-way valve 718 or by actuating a relief valve or vent. The refilling of the reservoir 700 may be performed during a procedure or between procedures, as necessary. The water may be sterile or non-sterile, as desired. For example, sterile water may be used for therapeutic procedures while non-sterile water may be used for diagnostic procedures. It is contemplated that refilling the reservoir 700 with sterile or non-sterile water may create more flexibility and reduce the need to have as much sterile water in storage. Further, refilling the reservoir 700 via the port 724 and removable cap 726 may also remove the need to disconnect the reservoir 700 from the tubing 712, 720 throughout the day eliminating or greatly reducing the possibility of cross contamination by removing the need to replace the water container.

FIG. 9A depicts a perspective view of an illustrative manifold 800 configured to couple a lens wash or shared water supply tube and a gas or shared gas supply tube to a water reservoir, such as those disclosed herein. FIG. 9B depicts an alternative perspective view of the illustrative manifold 800 of FIG. 9A. FIG. 9C depicts a cross-sectional view of the illustrative manifold 800 of FIG. 9A, taken at line 9C-9C, and including a shared gas supply tube 806 and water supply tube 808. As used herein, a “manifold” is a structure having three or more openings for making fluid connections, with the manifold 800 comprising a structure that couples two or more tubes to a reservoir at a single reservoir opening. The manifold 800 may extend from a first end 802 configured to be positioned within an interior of the reservoir or container to a second end 804 configured to be positioned exterior to the reservoir or container. The second end 804 of the manifold may include an elongate tube or port 810 defining an inlet 826 for receiving a flow of gas and/or water. The elongate tube 810 may include a plurality of barbs or raised portions 812. The barbs 812 may increase in diameter towards the first end 802 of the manifold 800. The barbs 812 may be configured to facilitate placement of the gas supply tube 806 over the elongate tube 810 (e.g., toward the first end 802) while limiting retraction or uncoupling of the gas supply tube 806.

The manifold 800 may be connected in fluid communication with a tubing manifold (not explicitly shown) via a shared gas supply/alternate gas supply tubing (or gas supply tubing) 806 and a lens wash supply/irrigation supply tubing or water supply tubing 808. The shared gas supply tubing 806 extends from a second end external to the reservoir to a first end coupled to the elongate tube 810 and/or first inlet 826 of the manifold 800. Air travels through a lumen 814 of the gas supply tubing 806 through a lumen 816 of the elongate tube 810, and into a cavity 818 of a body 820 of the manifold 800. Air exits the cavity through a first air outlet 828 (FIG. 9B). The first air outlet 928 may include a plurality of apertures 830a-e (collectively, 830). While the first air outlet 928 is shown and described as having five apertures 830, the first air outlet 928 may have fewer than five or more than five apertures, as desired.

A one-way valve 832 (FIG. 9C) may positioned in or adjacent to the first air outlet 828. In some examples, the one-way valve 832 may be a flap valve although other one-way valves, including those described elsewhere herein, may be used, as desired. The one-way valve 832 may be configured to allow air to move from the cavity 818 of the manifold 800 and exit the manifold as shown at arrows 834. However, the one-way valve 832 may prevent air from moving in the opposite direction. This may allow air to enter and pressurize the reservoir. This may also prevent water from entering the cavity 818 of the body 820 of the manifold 800. The one-way valve 832 may be coupled to the first air outlet 828 using a number of techniques including, but not limited to, glue, adhesives, sonic welding, ultrasound welding, etc.

A lens wash supply tube or shared water supply tube (e.g. supplies water for both lens wash and irrigation) 808 extends from a second end external to the manifold 800 to a first end which extends into the reservoir such that the water supply tubing 808 is in operative fluid communication with an interior or a cavity of the reservoir. A lumen 824 extends through the water supply tubing 808 for receiving a flow of fluid therethrough. The water supply tube 808 may extend coaxially through a lumen 814 of the gas supply tube 806 and coaxially through a lumen 816 of the elongate tube 810. The water supply tube 808 may further extend through the cavity 818 in the body 820 of the manifold 800 to first fluid outlet 822 adjacent the first end 802 of the manifold 800. The water supply tube 808 may extend through the first fluid outlet 822 and into the interior of a reservoir (not explicitly shown) to place the lumen 824 of the water supply tube 808 in fluid communication with a fluid in the reservoir. In where the manifold is placed a bottom portion of the reservoir, no extension of the water supply tube 808 beyond the first fluid outlet 822 is necessary.

The manifold 800 may further include one or more laterally extending protrusions or shoulders 836a, 836b (collectively, 836). The shoulders 836 may be configured to extend along an inner surface of the reservoir while the body portion 820 extends through a wall of the reservoir such that the body portion 820 and the elongate tube 810 are positioned exterior to the reservoir. For example, a cut may be made in a seam of the reservoir (or at another desired location) and the manifold 800 inserted therethrough. The manifold 800 may then be secured to the reservoir. In some embodiments, the shoulders 836 may be adhered, glued, or otherwise affixed to an inner surface of the reservoir to provide a fluid-tight seal. In some embodiments, the shoulders 836 may terminate in a point 838a, 838b which may decrease the stress on the seam of the reservoir.

As described herein, in some cases, the reservoirs 500, 600, 700 may be fixed relative to the endoscope tower. While the coupling mechanisms are described with respect to a reservoir 500, 600, 700, it is contemplated that other devices, equipment, components, etc. may be coupled to the endoscope tower using the coupling mechanisms described herein. It is contemplated that there are many features on a standard endoscope tower that the reservoir 500, 600, 700 could be coupled to including, but not limited to, endoscope hanging features, hand hold features, closed loop features, IV bag towers, flat shelving features, monitor arms, thin brackets, etc. The reservoir 500, 600, 700 can be affixed to these features either directly, using compatible sections built into the reservoir 500, 600, 700, or indirectly, by using additional fixtures such as clasps, hooks, or other tools. These features/fixtures on the reservoir could come in the form of hanging hooks, magnets (if the tower is metallic in nature), zip tie features, or holes for any hanging hook. Alternatively, the reservoir 500, 600, 700 itself could be designed to fit on universal tower shelves without taking up much space, allowing for it on a shelf with a piece of capital still existing on it by fitting in front of or to the side of the capital. In some cases, the reservoir 500, 600, 700 may be mounted relative to or placed within a drawer that is mounted on a tower shelf. This drawer may slide in and out and have an opening for users to fill the reservoir. In yet other examples, a hook and loop fastener could also be installed on the side of a shelf and come with every reservoir 500, 600, 700. This may allow the user to connect the reservoir 500, 600, 700 quickly and easily to the side of the shelf via the hook and loop strips. Additional shelves or horizontal surfaces could be coupled to the tower on the front, back or side to specifically house the reservoir of water.

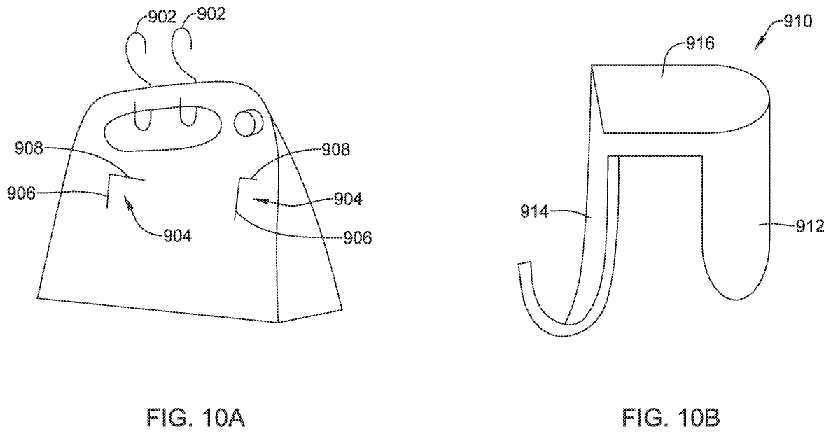

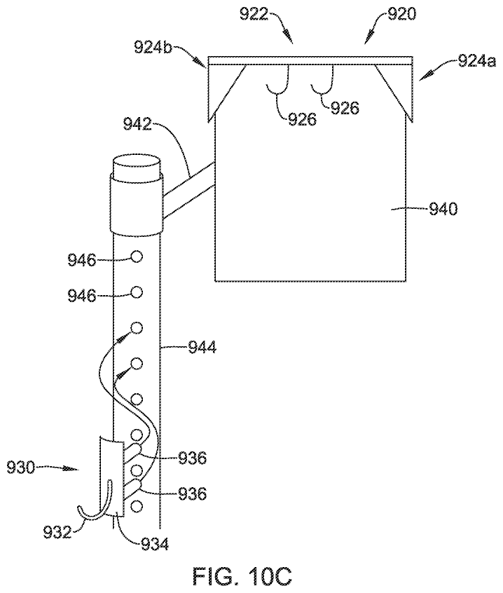

FIG. 10A depicts two different ways for securing a reservoir 500, 600, 700 relative to an endoscope tower. In a first example, “S” hooks 902 may be used to hang the reservoir 500, 600, 700 from a hook, a bar, a shelf, etc. of the endoscope tower. A first portion of the “S” hook 902 may be received through handle in the reservoir 500, 600, 700 while a second portion may fit into one of the endoscope-hanging hole features on an endoscope tower. This may allow the user to hang the reservoir 500, 600, 700 at an ergonomic height for the users. In another example, “L” brackets 904 may be secured to or incorporated into the reservoir 500, 600, 700. The “L” brackets 904 may have a first leg 906 secured relative to the reservoir 500, 600, 700 and a second leg 908 extending generally orthogonal to the first leg 906 and configured to be received in an aperture on the endoscope tower. In some embodiments, the second leg 908 may extend at non-orthogonal angles relative to the first leg 906.

FIG. 10B depicts a perspective view of another illustrative coupling mechanism 910 for securing a reservoir 500, 600, 700 relative to an endoscope tower. The coupling mechanism 910 may include a post portion 912, a hook portion 914, and a body portion 916 extending between the post portion 912 and the hook portion 914. The post portion 912 may be sized and shaped to be received in an opening of the endoscope tower, such as, but not limited to, a handle hole. The hook portion 914 may be received through handle in the reservoir 500, 600, 700.

FIG. 10C depicts a perspective view of additional coupling mechanisms 920, 930. Endoscope towers may have monitors 940 at the top of the cart attached to a post 944 via an arm 942. In one example, a coupling mechanism 920 may include a bracket 922 extending between a first corner bracket 924a and second corner bracket 924b (collectively, 924). The corner brackets 924 may be configured to be placed over the corners of the monitor 940. One or more hooks 926 may be fixedly or releasably secured to the bracket 922 and configured to extend along a backside or a lateral side of the monitor 940 so as not to hinder visibility of the display. The hooks 926 may be received through handle in the reservoir 500, 600, 700.

In some embodiments, the height of the monitor 740 is adjustable via pin holes 946 in the pole 944. It is contemplated that a coupling mechanism 930 may utilize the pin holes 946 to secure a reservoir 500, 600, 700 to the post 944. For example, the coupling mechanism 930 may include a hook 932 coupled to a first side of a plate 934. One or more pins 936 may extend from a second side of the plate 934 opposite the first side. The one or more pins 936 may be received in the pin holes 946 of the post 944. In some embodiments, the one or more pins 936 may form a press-fit with the pin holes 946. Alternatively or additionally, a strap may be used to further secure the coupling mechanism 930 to the post 944.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container arranged and configured to couple to an endoscope for use in an endoscopic procedure, the container comprising:
a container configured to contain a fluid, the container having a bottom portion and a top portion;
a manifold including a first end positioned interior to the container and a second end positioned exterior to the container, the manifold configured to couple a water supply tube and a gas supply tube to the container; and
a port positioned adjacent to the top portion of the container, wherein the port is configured to be in selective fluid communication with an interior of the container.

2. The container of claim 1, wherein the first end of manifold comprises an air outlet and a fluid inlet.

3. The container of claim 2, wherein a first lumen of the water supply tube is fluidly coupled with the fluid inlet.

4. The container of claim 2, wherein the air outlet comprises a one-way valve.

5. The container of claim 2, wherein the second end of the manifold comprises an air inlet.

6. The container of claim 5, wherein a first end of the gas supply tube is coupled to the air inlet of the manifold.

7. The container of claim 5, wherein the water supply tube is configured to extend coaxially through the air inlet of the manifold to the fluid inlet.

8. The container of claim 1, wherein the manifold further comprises at least one laterally extending protrusion.

9. The container of claim 8, wherein the at least one laterally extending protrusion is positioned interior to the container.

10. The container of claim 8, wherein the at least one laterally extending protrusion is adhered to the interior of the container.

11. The container of claim 1, further comprising a cap removably coupled to the port.

12. The container of claim 1, wherein the water supply tube includes a first end, a second end, and a first lumen extending therethrough, wherein the first lumen is in fluid communication with the bottom portion of the container and the second end of the water supply tube is positioned external to the container; and
the gas supply tube includes a first end, a second end, and a second lumen extending therethrough, wherein the second lumen is in operative fluid communication with container and the second end of the gas supply tube is positioned external to the container.

13. A container and tube set arranged and configured to couple to an endoscope for use in an endoscopic procedure, the container and tube set comprising:
a vessel;
a first chamber disposed within the vessel and configured to contain a fluid;
an inflatable bladder having an inlet and disposed within the vessel;
a water outlet in fluid communication with the first chamber;
a gas inlet in fluid communication with the inflatable bladder; and
a port positioned adjacent to the top portion of the first chamber, wherein the port is configured to be in selective fluid communication with an interior of the first chamber.

14. The container and tube set of claim 13, wherein the water outlet comprises a water supply tube including a first end, a second end, and a first lumen extending therethrough, wherein the first lumen is in fluid communication with the first chamber and the second end of the water supply tube is positioned external to the vessel; and
wherein the gas inlet comprises a gas supply tube including a first end, a second end, and a second lumen extending therethrough, wherein the second lumen is in operative communication with the inlet of the inflatable bladder and the second end of the gas supply tube is positioned external to the container.

15. The container and tube set of claim 13, further comprising a one-way valve disposed in the inlet of the inflatable bladder.

16. The container and tube set of claim 15, wherein the one-way valve is removable.

17. The container and tube set of claim 13, wherein the inflatable bladder is configured to expand to expel fluid from the first chamber.

18. The container and tube set of claim 13, further comprising a cap removably coupled to the port.

19. The container and tube set of claim 18, wherein the cap comprises a tapered plug, a self-sealing cap, or a self-sealing valve.

20. A container arranged and configured to couple to an endoscope for use in an endoscopic procedure, the container comprising:
a container configured to contain a fluid, the container having a bottom portion and a top portion;
a water supply tube including a first end, a second end, and a first lumen extending therethrough, wherein the first lumen is in fluid communication with the bottom portion of the container and the second end of the water supply tube is positioned external to the container;
a gas supply tube including a first end, a second end, and a second lumen extending therethrough, wherein the second lumen is in operative fluid communication with the container and the second end of the gas supply tube is positioned external to the container; and
a port positioned adjacent to the top portion of the container, wherein the port is configured to be in selective fluid communication with the container and wherein the port is configured to couple directly to a water bottle, the coupling between the port and water bottle free from tubes and/or conduits.
